(19) United States
(12) Patent Application Publication
(10) Pub. No.: US 2006/0235459 A1
(43) Pub. Date: Oct. 19, 2006

(54) Balloon catheters and methods for manufacture

(76) Inventor: Gladwin S. Das, Arden Hills, MN (US)

Correspondence Address: CYR & ASSOCIATES, P.A., PONDVIEW PLAZA, 5850 OPUS PARKWAY SUITE 114, MINNETONKA, MN, 55343, US

(21) Appl. No.: 11/109,335

(22) Filed: Apr. 19, 2005

(51) Int. Cl. A61M 29/00

(52) U.S. Cl. 606/192

(57) ABSTRACT

A balloon catheter and stent delivery system for medical treatment of a patient are provided. The balloon catheter includes a hub, a shaft and a Y-shaped balloon. The Y-shaped balloon may include a first distal arm and a second distal arm having different diameters. The Y-shaped balloon may also include a first elongated balloon positioned or secured to a second elongated balloon to form the Y-shaped balloon. The first and second elongated balloons may be balloons having a circular profile at their distal end and a semi-circular profile at their proximal ends.

### BACKGROUND OF THE INVENTION

1. Field of the Invention

The present invention relates to stents and, more particularly, to apparatus and methods for the implantation of bifurcated stents.

2. Description of the Related Art

Balloon catheters are used in a variety of therapeutic applications, including intravascular catheters for procedures such as angioplasty. By way of example, the present invention will be described in relation to coronary and peripheral angioplasty treatments. However, the present invention relates to any balloon catheter and stent delivery system having enhanced stent retention, and is not limited to angioplasty.

Most balloon catheters have an elongated flexible shaft defining one or more passages or lumens with one or more inflatable balloons attached near or at one end of the shaft. For reference, the end of the catheter including the balloon will be referred to as the “distal” end, while the other end is called the “proximal” end. The relative position of various components may also be referred to as “proximal” and “distal” based on their position generally along the longitudinal axis of the shaft or more generally based on the distance from the proximal end when the shaft is straightened out to a substantially linear configuration. The balloons may be connected to one or more inflation lumen extending through the shaft for the purpose of selectively inflating and deflating the balloon. The other end of the inflation lumen and other lumen within the shaft may be in fluid communication with a hub to couple the lumen to various devices.

One method for using a balloon catheter is to advance its distal end into the body of a patient, by directing the distal end of the balloon catheter percutaneously through an incision and into a body passage such as a blood vessel. The distal end of the balloon catheter is advanced until the balloon is positioned at a target location. After the balloon is disposed within the target location, the balloon may be inflated to press outward on the body passage. The pressure may be relatively high pressure when the material from which the balloon is formed is an inelastic or non-compliant.

This outward pressing of a constriction or narrowing at the desired site in a body passage is intended to partially or completely re-open or dilate that body passageway or lumen, increasing its inner diameter or cross-sectional area. In the case of a blood vessel, this procedure is referred to as angioplasty. The objective of an angioplasty is to increase the inner diameter or cross-sectional area of the vessel passage or lumen to allow blood to flow more easily through the effected region. The narrowing of the body passageway lumen is typically called a lesion or stenosis, and may be in the form of hard plaque or viscous thrombus.

Unfortunately, the lumen at the angioplasty site may re-close or become narrow again. This will typically occur at around six months after the angioplasty procedure. This narrowing phenomenon is called restenosis. Restenosis occurs in as many as 30-40% of percutaneous transluminal angioplasty patients. Restenosis may require additional procedures, such as another angioplasty, drug therapy treatment, or even surgery including bypass graft. It is generally desirable to prevent or limit the occurrence of restenosis. This is particularly the case in patients whose poor health or other conditions may not make them the preferred candidates for repeated interventional treatment.

In an effort to prevent restenosis, short flexible cylinders or scaffolds made of metal or polymers, referred to as a stent, may be permanently implanted into the vessel to hold the lumen open, to reinforce the vessel wall and improve blood flow. Stents tend to keep the blood vessel open longer. The efficacy of stents has been improved in recent years by the addition of drug coatings which inhibit restenosis as well as other problems associated with the implantation of stents. Unfortunately, the use of stents can be limited by various factors, including size and location of the blood vessel, a complicated or tortuous vessel pathway, bifurcations in the blood vessels etc.

Some stents are expanded to the proper size by inflating a balloon catheter, referred to as “balloon-expandable” stents, while others are designed to elastically resist compression in a “self-expanding” manner. Both balloon-expandable stents and self-expanding stents are generally crimped or compressed to a diameter during delivery that is smaller than the eventual deployed diameter at the desired site. When positioned at the desired site within the lesion, they are deployed by inflating a balloon or being allowed to self-expand into the desired diameter.

### SUMMARY OF THE INVENTION

The present inventions satisfy needs and provide improvements and advantages in the area of balloon catheters that will be recognized by those skilled in the art upon review of the present disclosure.

In one aspect, the present invention provides a Y-shaped balloon for a balloon catheter. The Y-shaped balloon may be formed from the relative positioning of a first elongated balloon and a second elongated balloon. The first elongated balloon can include a first semi-circular region and a first circular region. The first semi-circular region may extend over at least a portion of the first proximal end of the first elongated balloon. The first semi-circular region may extend over at least a portion of the first distal end of the first elongated balloon. The first circular region defines a first diameter. The second elongated balloon can include a second semi-circular region and a second circular region. The second semi-circular region can extend over at least a portion of the second proximal end of the second elongated balloon. The second circular region may extend over at least a portion of a second distal end of the second elongated balloon. The second circular region can define a second diameter. In one aspect, the second diameter of the second circular region may be greater than the first diameter of the first circular region. In another aspect, the second diameter may be substantially equal to a proximal diameter. In another aspect, at least a portion of the first semi-circular region is secured to at least a portion of the second semicircular region to form a circular proximal region. In another aspect, the first semi-circular region may be secured to the second semicircular region by at least a first joint. In another aspect, the first semi-circular region may be secured to the second semicircular region by a first joint and a second joint. In another aspect, the first semi-circular region may be sealingly secured to the second semicircular region. In another aspect, the Y-shaped balloon for a balloon catheter may include at least a first inner tube extending from a proximal end of the first elongated balloon to a distal end of the first elongated balloon. In still another aspect, the Y-shaped balloon may include at least a second inner tube extending from a proximal end of the second elongated balloon to a distal end of the second elongated balloon. A stent may be secured over at least a portion of the Y-shaped balloon.

In another aspect, the present invention may include a balloon catheter having a hub, a shaft and a Y-shaped balloon. The shaft may have a proximal end and a distal end. The proximal end may be secured to the hub and the distal end may be secured to a Y-shaped balloon. The Y-shaped balloon may have a proximal arm, a first distal arm and a second distal arm. The proximal arm may define a proximal diameter. The first distal arm may define a first distal diameter. The second distal arm may define a second distal diameter. In one aspect, the proximal diameter and the second distal diameter may be substantially equivalent. In another aspect, the proximal diameter is at least 1.2 times larger than the first distal diameter. In another aspect, the shaft defining an inflation lumen in fluid communication with an interior chamber of the Y-shaped balloon.

### BRIEF DESCRIPTION OF THE FIGURES

FIG. 1 illustrates a perspective view of an embodiment of a balloon catheter in accordance with the present invention;

FIG. 2 illustrates a cross section of an embodiment of a Y-shaped balloon in accordance with the present invention in a deflated configuration;

FIG. 3 illustrates a cross section of an embodiment of a Y-shaped balloon in accordance with the present invention in a deflated configuration in at least a partially inflated configuration;

FIG. 4 illustrates a diagrammatic side view of an embodiment of a Y-shaped balloon in accordance with the present invention in at least a partially inflated configuration;

FIG. 5 illustrates a diagrammatic side view of another embodiment of a Y-shaped balloon in accordance with the present invention in at least a partially inflated configuration;

FIG. 6 illustrates a cross-section through an embodiment of the proximal arm of a Y-shaped balloon in accordance with the present invention in at least a partially inflated configuration;

FIG. 7 illustrates a cross-section through another embodiment of the proximal arm of a Y-shaped balloon in accordance with the present invention in at least a partially inflated configuration;

FIG. 8 illustrates a cross-section through another embodiment of the proximal arm of a Y-shaped balloon in accordance with the present invention in at least a partially inflated configuration; and

FIG. 9 illustrates a cross-section through another embodiment of the proximal arm of a Y-shaped balloon in accordance with the present invention in at least a partially inflated configuration.

All Figures are illustrated for ease of explanation of the basic teachings of the present invention only; the extensions of the Figures with respect to number, position, proportions, relationship and dimensions of the parts to form the illustrated embodiments will be explained or will be within the skill of the art after the following description has been read and understood. Further, the exact dimensions and dimensional proportions to conform to specific force, weight, strength, and similar requirements will likewise be within the skill of the art after the following description has been read and understood.

Where used in various Figures of the drawings, the same numerals designate the same or similar parts. Furthermore, when the terms “top,” “bottom,” “right,” “left,” “forward,” “rear,” “first,” “second,” “inside,” “outside,” and similar terms are used, the terms should be understood to reference only the structure shown in the drawings as it would appear to a person viewing the drawings and utilized only to facilitate describing the illustrated embodiment.

### DETAILED DESCRIPTION OF THE INVENTION

Aspects of a balloon catheter system, a balloon catheter and Y-shaped balloon in accordance with the present invention are illustrated in exemplary embodiments throughout the attached figures. A balloon catheter in accordance with the present invention has been generally designated “10”.

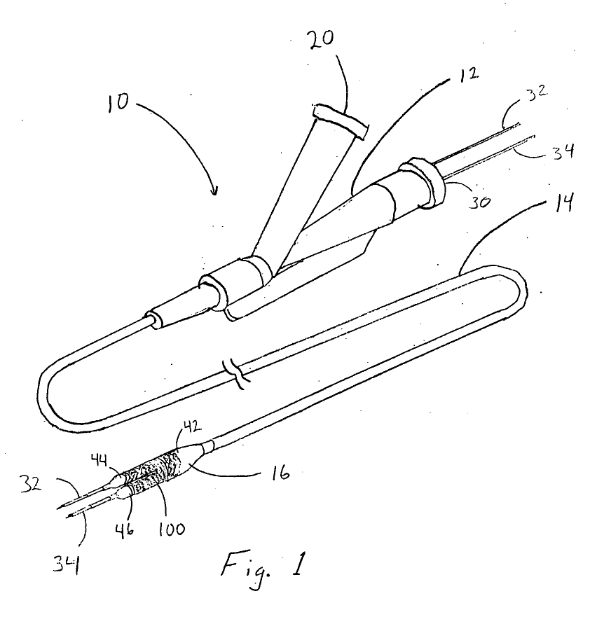

As illustrated in FIG. 1, a balloon catheter 10 in accordance with the present invention may include a hub 12, an elongated and flexible tubular shaft 14, and an inflatable Y-shaped balloon 16. The Y-shaped balloon 16 is typically secured to the shaft 14 near a distal end of the shaft 14, and the hub is affixed to the proximal end of the shaft 14.

The shaft 12 permits the controlled positioning of the Y-shaped balloon 16 as well as a conduit for communication with the Y-shaped balloon 16 while the Y-shaped balloon 16 is within a patient. The shaft 12 may be configured to be steerable or otherwise directable by a user as will be recognized by those skilled in the art. The steerability or directability of the shaft simplifies navigation through a passage or lumen within a patient. The shaft may include electronics or fiber optics (not shown) to transmit images or other information from within the patient to a user. The shaft 14 can define one or more passages or lumens extending through the shaft 14, one of which may be an inflation lumen 18. The distal end of the inflation lumen 18 is typically in fluid communication with an interior 22 of the Y-shaped balloon 16. Thus, the inflation lumen 18 may provide for fluid communication between the interior 22 of the Y-shaped balloon 16 at the distal end of the inflation lumen 18. The proximal end of the inflation lumen 18 is typically in fluid communication with a pump or other source of pressurized fluid for the purpose of inflating the Y-shaped balloon 16. In one aspect, the pressurized fluid may be dry nitrogen.

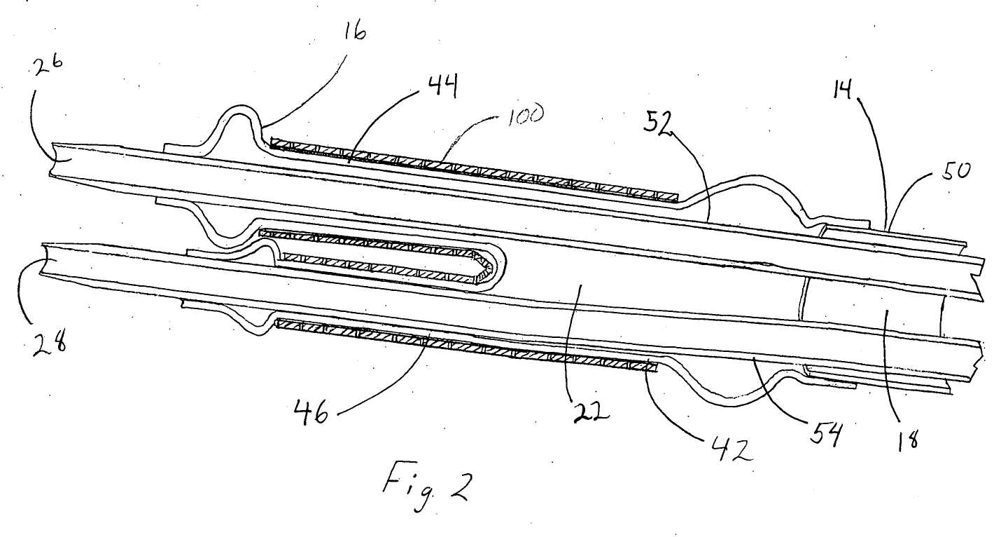

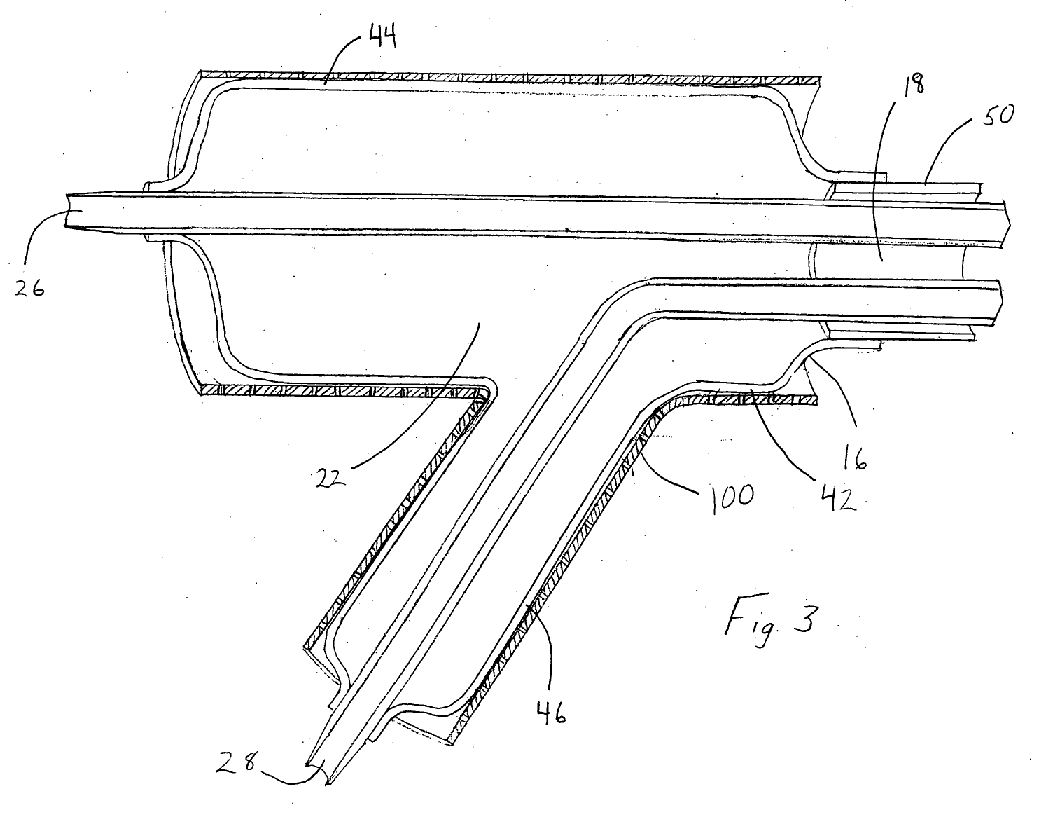

The hub 12 is affixed to the proximal end of the shaft 14. The hub 12 may generally form the point of articulation for a user of the balloon catheter 10. The shaft 14 is generally secured to or within the hub 12. The hub 12 and adjacent shaft portion may include a tubular strain relief. The hub 12 of a balloon catheter 10 typically includes an inflation port 20 having a coupling, such as a luer-lock type fitting, for connecting the inflation lumen 18 to a source of pressurized fluid. The hub 12 also typically includes one or more guidewire ports 30. The guidewire ports 30 may be in communication with guidewire lumen 26, 28, defined by the shaft 14, shown in FIGS. 2 and 3, to receive one or more guidewires 32, 34 over which the balloon catheter 10 is passed during a procedure to position the Y-shaped balloon 16 within a patient. The guidewire ports 30 may include hemostatic valves. Such a valves can allow the guidewire to traverse and slide within the guidewire lumen 26, 28, while resisting the loss of blood or other fluids through the guidewire lumen 26, 28 and guidewire ports 30.

The Y-shaped balloon 16 is generally configured to expand from an uninflated position to an inflated position. The Y-shaped balloon may be constructed of a variety of different materials, including for example Nylon, PEEK, Pebax, among others. The Y-shaped balloon generally includes a proximal arm 42, a first distal arm 44 and a second distal arm 46. In one aspect, the Y-shaped balloon 16 may include a bifurcated stent 100 or at least two separate stents 100 secured about the outer surface of the Y-shaped balloon 16. The bifurcated stent 100 or the at least two separate stents 100 are configured to be deployed at a vessel bifurcation may be secured over the un-inflated Y-shaped balloon 16. A bifurcated stent 100 may generally secured such that the branches of the bifurcated stent 100 are received over the proximal arm 42, the first distal arm 44 and the second distal arm 46 of the Y-shaped balloon 16. When the Y-shaped balloon 16 is inflated the bifurcated stent 100 may be expanded and will typically remain expanded after the Y-shaped balloon 16 is deflated. Similarly, at least two separate stents 100 may generally secured such that each of the proximal arm 42, the first distal arm 44 and the second distal arm 46 of the Y-shaped balloon 16 includes one of the at least two separate stents 100. When the Y-shaped balloon 16 is inflated each of the at least two separate stents 100 may be expanded and will typically remain expanded after the Y-shaped balloon 16 is deflated. Accordingly, the bifurcated stent 100 or the at least two separate stents 100 may be retained the vessel in an open position after the Y-shaped balloon is deflated and removed from the target location.

As illustrated for exemplary purposes in FIGS. 2 and 3, the shaft 14 may be constructed of an outer tube 50, first inner tube 52, and a second inner tube 54. First inner tube 52 and second inner tube 54 may extend through the entire length of the shaft and Y-shaped balloon 16. As illustrated, the first inner tube 52 and the second inner tube 54 extend through the Y-shaped balloon and form the distal end of the balloon catheter 10. The distal portions of first distal arm 42 and second distal arm 44 are sealingly secured about the first inner tube 52 and the second inner tube 54 to permit the Y-shaped balloon 16 to expand upon receiving a fluid through inflation lumen 18 of shaft 50. The first inner tube 52 and the second inner tube 54 define a first guidewire lumen 26 and a second guidewire lumen 28, respectively. The guidewire lumen 26, 28 are generally adapted to receive one or more elongated flexible guidewires 32, 34 in a sliding fashion, such that the guidewire 32, 34 and balloon catheter 10 may be advanced or withdrawn independently, or the balloon catheter 10 may be guided along a path selected with the guidewire 32, 34. The shaft 14 may of course have various configurations instead of this coaxial design, including a single extruded tube defining any suitable number of parallel or spiraling side-by-side lumens, among other configurations. The inflation lumen 18 is defined by the annular space between the inner tubes 52, 54 and outer tube 50 in the illustrated embodiment. The inflation lumen 18 communicates between the interior 22 of the Y-shaped balloon 16 and a source of pressurized fluid. Accordingly, Y-shaped balloon 16 is typically sealing secured about inflation lumen 18. The Y-shaped balloon is typically secured to shaft 14. As illustrated, the Y-shaped balloon is secured to an exterior surface of the outer tube 50 of shaft 14 to permit the inflation aperture 56 of Y-shaped balloon 16 to be in fluid communication with the inflation lumen 18 of shaft 14. The pressurized fluid is received from the pump or other source and conveyed through the inflation lumen 18 to the interior chamber 22 of the Y-shaped balloon 16.

FIG. 2 illustrates a Y-shaped balloon 16 in an un-inflated configuration. As illustrated, a bifurcated stent 100 is secured about the uninflated Y-shaped balloon 16. The assembly of the bifurcated stent 100 and the Y-shaped balloon 16 is in a substantially linear configuration to permit its introduction through a lumen or passage in the patient. Once positioned over on or more guidewires 32, 34 within a patient, a pressurized fluid is introduced into the interior 22 of the Y-shaped balloon 16. This tends to inflate the balloon by expanding the material of the Y-shaped balloon 16, unfolding pleats in the material of the Y-shaped balloon 16, or by a combination thereof.

FIG. 3 illustrates a Y-shaped balloon 16 similar to the embodiment of FIG. 2 in an at least partially inflated configuration. As illustrated, the bifurcated stent 100 has been biased into an operable position by the expansion of the Y-shaped balloon 16. The Y-shaped balloon 16 may be removed from the patient once it is deflated.

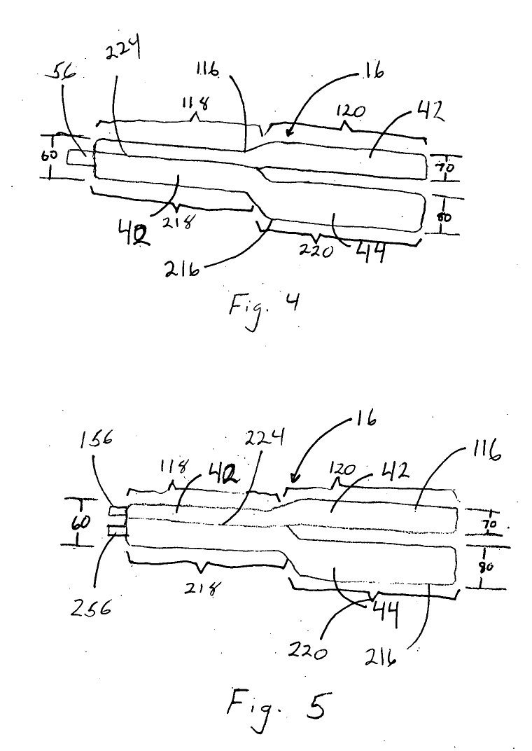

FIGS. 4 and 5 diagrammatically illustrate two exemplary embodiments of a Y-shaped balloon 16 including aspects of the present invention. The Y-shaped balloons 16 of FIGS. 4 and 5 include a first elongated balloon 116 and a second elongated balloon 216 secured to one another at their proximal ends. The first elongated balloon 116 includes a first semi-circular region 118 at a first proximal end and a first circular region 120 at a first distal end. The second elongated balloon 216 includes a second semi-circular region 218 at a second proximal end and a second circular region 220 at a second distal end. The first semi-circular region 118 of the first elongated balloon 116 is secured to the second semi-circular region 218 of the second elongated balloon 216 to form a Y-shaped balloon 16. FIG. 4 illustrates a Y-shaped balloon 16 having a single inflation aperture 56 in communication with the first interior 122 and second interior 222 (shown in FIGS. 6 and 7) of the first elongated balloon 116 and the second elongated balloon 216, respectively. FIG. 5 illustrates a Y-shaped balloon 16 having a first inflation aperture 156 in communication with the first interior 122 of the first elongated balloon 116 and a second inflation aperture 256 in fluid communication with the second interior 222 of the second elongated balloon 216, as would be operable in the embodiments shown in FIGS. 6 and 9.

As illustrated in FIGS. 1 to 5, a Y-shaped balloon 16 in accordance with the present invention may be configured with the proximal arm 42 having a proximal diameter 60, the first distal arm 44 having a first distal diameter 70, and the second distal arm 46 having a second distal diameter 80. Each of the proximal diameters 60, first distal diameter 70 and second distal diameter 80 represents the maximum diameter of the operatively inflated respective arm of the Y-shaped balloon 16.

In an aspect of the present invention, the proximal diameter 60 may be equal to or substantially equal to the second distal diameter 80. In this aspect, the proximal diameter 60 is greater than the first distal diameter 70. The proximal diameter 60 can be at least 1.2 times larger than the first distal diameter 70.

In another aspect of the present invention, the proximal diameter 60 is greater than the second distal diameter 80. In this aspect, the second distal diameter 80 is greater than the first distal diameter 70. The proximal diameter 60 is at least 1.2 times larger than the first distal diameter 70.

In yet another aspect of the present invention, the proximal diameter 60 is greater than the first distal diameter 70. In this aspect, the first distal diameter 70 is greater than the second distal diameter 80. The proximal diameter 60 is at least 1.2 times larger than at least one of the first distal diameter 70 and the second distal diameter 80.

FIGS. 6 to 9 illustrate a transverse cross-section through the first semi-circular region 118 of the first elongated balloon 116 and the second semi-circular region 218 of the second elongated balloon 216. The first semi-circular region 118 is secure to the second semi-circular region 218 to form a Y-shaped balloon 16. In one aspect, the first semi-circular region 118 and the second semi-circular region 218 may be secured to form a substantially circular cross-section as is generally illustrated in FIGS. 6 to 9. As used herein, “circular” is not used in the geometric sense of a perfect circle but instead as a functional approximation of a circular shape which may permit a Y-shaped balloon catheter to expand a stent into an operable position or to open a clogged artery such as in a balloon angioplasty procedure. Further, “semi-circular” is used to approximate any fractional portion of a circle. In yet another aspect, the first semi-circular region 118 and the second semi-circular region 218 are secured to one another to create a seal. The first semi-circular region 118 and the second semi-circular region 218 may be secured to one another by welding, adhesives, or otherwise secured to one another as will be recognized by those skilled in the art upon review of the present disclosure. When secured to one another, a weld or adhesive will typically form at least a first exterior joint 124 and a second exterior joint 224 along the periphery of the first semi-circular region 118 and the second semi-circular region 218. The first exterior joint 124 and the second exterior joint 224 may form a seal along the interface between the first semi-circular region 118 and the second semi-circular region 218 to sealingly secure the first elongated balloon 116 to the second elongated balloon 216. In one aspect, the seal created by first exterior joint 124 and the second exterior joint 224 may extend about the entire interface between the first semi-circular region 118 and the second semi-circular region 218 such that the first exterior joint 124 and the second exterior joint 224 may meet at both the distal and proximal portions of the first semi-circular region 118 and the second semi-circular region 218. That is, the first joint 124 and the second joint 224 together could extend circumferentially about a longitudinal cross-section through the first semi-circular region 118 and the second semi-circular region 218. In still another aspect, the first semi-circular region 118 and the second semi-circular region 218 are merely positioned adjacent to one another without being secured to one another. Depending on the particular profiles of first semi-circular region 118 and second semi-circular region 218, a proximal arm 42 including a first semi-circular region 118 and a second semi-circular region 218 may have a substantially circular profile in transverse profile as noted above.

The first semi-circular region 118 and the second semi-circular region 218 may have a number of configurations of which some exemplary configurations are illustrated in FIGS. 6 to 9.

FIG. 6 illustrates an embodiment of the first semi-circular region 118 and the second semi-circular region 218 which are sealingly secured to one another by a first joint 124 and second joint 224. The first semi-circular region 118 and the second semi-circular region 218 do not include material extending through the longitudinal plane to independently define first interior 122 and second interior 222 of the respective first elongated balloon 116 and the second elongated balloon 216. Accordingly, a single interior space 22 similar to that of FIGS. 1 to 3 is defined by the embodiment of FIG. 6. As illustrated, the first joint 124 and the second joint 224 will sealingly interconnect the first semi-circular region 118 and the second semi-circular region 218 to permit the Y-shaped balloon 16 to be inflated. Such an embodiment would only require a single inflation aperture 56 to permit both the first elongated balloon 116 and second elongated balloon 216 which form the Y-shaped balloon 16 to be inflated.

FIG. 7 illustrates an embodiment of the first semi-circular region 118 and the second semi-circular region 218 which are secured to one another by a first joint 124 and second joint 224. As illustrated, the first semi-circular region 118 and the second semi-circular region 218 include material extending through the longitudinal plane to distinctly define first interior 122 and second interior 222 of the respective first elongated balloon 116 and the second elongated balloon 216. As illustrated for exemplary purposes, both the first semi-circular region 118 and the second semi-circular region 218 include the material extending through the longitudinal plane. The first interior 122 and second interior 222 are illustrated as fluidly interconnected by a interconnection aperture 138 defined in the material extending through the longitudinal plane of at least one of the first semi-circular region 118 and the second semi-circular region 218. As illustrated, the first joint 124 and the second joint 224 interconnect the first semi-circular region 118 and the second semi-circular region 218 to permit the Y-shaped balloon 16 to be inflated. The first joint 124 and the second joint 224 may sealingly interconnect the first semi-circular region 118 and the second semi-circular region 218. However, the longitudinal material of the first semi-circular region 118 and the second semi-circular region 218 may be sealingly secured to one another only about the interconnection aperture 138 so that the first joint 124 and the second joint 224 do not need to form a seal. Because of the interconnection aperture, the embodiment of FIG. 7 would only require a single inflation aperture 56 to permit both the first elongated balloon 116 and second elongated balloon 216 which form the Y-shaped balloon 16 to be inflated.

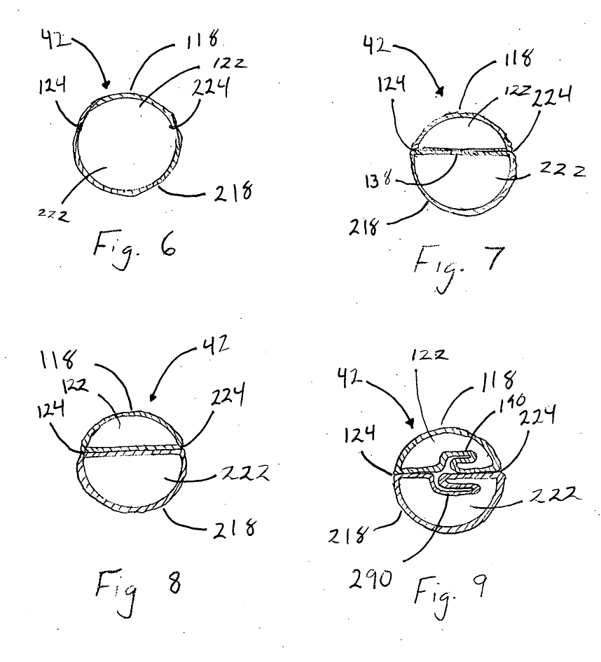

FIG. 8 illustrates an embodiment of the first semi-circular region 118 and the second semi-circular region 218 which may be secured to one another by a first joint 124 and second joint 224. Alternatively, the embodiment of the first semi-circular region 118 and the second semi-circular region 218 which may be merely positioned adjacent to one another to form a Y-shaped balloon 16. As illustrated, the first semi-circular region 118 and the second semi-circular region 218 include material extending through the longitudinal plane to distinctly define first interior 122 and second interior 222 of the respective first elongated balloon 116 and the second elongated balloon 216. As illustrated for exemplary purposes, both the first semi-circular region 118 and the second semi-circular region 218 include the material extending through the longitudinal plane. The first joint 124 and the second joint 224 may interconnect the first semi-circular region 118 and the second semi-circular region 218. The illustrated first interior 122 and second interior 222 may form independently sealed chambers which, in certain configurations, may be independently inflated and deflated. Because the first interior 122 and second interior 222 may form independently sealed chambers, the embodiment of FIG. 8 would typically require a first inflation aperture 156 and a second inflation aperture 256 to independently inflate and deflate the first elongated balloon 116 and second elongated balloon 216, respectively.

FIG. 9 also illustrates an embodiment of the first semi-circular region 118 and the second semi-circular region 218 which may be secured to one another by a first joint 124 and second joint 224. Alternatively, the illustrated embodiment of the first semi-circular region 118 and the second semi-circular region 218 which may again be merely positioned adjacent to one another to form a Y-shaped balloon 16 as noted in the description of the embodiment of FIG. 8. As illustrated, the first semi-circular region 118 and the second semi-circular region 218 include material extending through the longitudinal plane to distinctly define first interior 122 and second interior 222 of the respective first elongated balloon 116 and the second elongated balloon 216. As illustrated for exemplary purposes, both the first semi-circular region 118 and the second semi-circular region 218 include the material extending through the longitudinal plane.

In the illustrated embodiments, the first semi-circular region 118 and the second semi-circular region 218 are formed by folding a portion of a substantially circular portion of first elongated balloon 116 and second elongated balloon 216 inward to form a first folded portion 190 and a second folded portion 290 from the material extending through the longitudinal plane when the first elongated balloon 116 is secured second elongated balloon 216 at their proximal ends. The first joint 124 and the second joint 224 may interconnect the first semi-circular region 118 and the second semi-circular region 218 and may prevent the unfolding of the first folded portion 190 and the second folded portion 290 during inflation. The illustrated first interior 122 and second interior 222 may form independently sealed chambers which, in certain configurations, may be independently inflated and deflated. Because the first interior 122 and second interior 222 may form independently sealed chambers, the embodiment of FIG. 9 would typically require a first inflation aperture 156 and a second inflation aperture 256 to independently inflate and deflate the first elongated balloon 116 and second elongated balloon 216, respectively.

It should be understood that an unlimited number of configurations for the present invention could be realized. The foregoing discussion describes merely exemplary embodiments illustrating the principles of the present invention, the scope of which is recited in the following claims. Those skilled in the art will readily recognize from the description, claims, and drawings that numerous changes and modifications can be made without departing from the spirit and scope of the invention.

What is claimed is:

1. A Y-shaped balloon for a balloon catheter, comprising:
a first elongated balloon comprising a first semi-circular region over a first proximal end and a first circular region over a first distal end, the first circular region defining a first diameter; and
a second elongated balloon comprising a second semi-circular region over a second proximal end and a second circular region at a second distal end, the second circular region defining a second diameter, with at least a portion of the first semi-circular region secured to at least a portion of the second semicircular region to form a circular proximal region.

2. A Y-shaped balloon for a balloon catheter, as in claim 1, further comprising the second diameter greater than the first diameter.

3. A Y-shaped balloon for a balloon catheter, as in claim 2, further comprising the second diameter substantially equal to a proximal diameter.

4. A Y-shaped balloon for a balloon catheter, as in claim 1, further comprising the second diameter equal to a proximal diameter.

5. A Y-shaped balloon for a balloon catheter, as in claim 1, further comprising at least a first joint securing the first semi-circular region to the second semicircular region.

6. A Y-shaped balloon for a balloon catheter, as in claim 1, further comprising a first joint and a second joint securing the first semi-circular region to the second semicircular region.

7. A Y-shaped balloon for a balloon catheter, as in claim 6, further comprising the first semi-circular region sealingly secured to the second semicircular region.

8. A Y-shaped balloon for a balloon catheter, as in claim 1, further comprising at least a first inner tube extending from a proximal end of the first elongated balloon to a distal end of the first elongated balloon.

9. A Y-shaped balloon for a balloon catheter, as in claim 1, further comprising at least a second inner tube extending from a proximal end of the second elongated balloon to a distal end of the second elongated balloon.

10. A Y-shaped balloon for a balloon catheter, as in claim 1, further comprising a bifurcated stent secured over the first elongated balloon and the second elongated balloon.

11. A balloon catheter, comprising:
a hub;
a shaft comprising a proximal end and a distal end with the proximal end secured to the hub; and
a Y-shaped balloon secured to the distal end of the shaft, the Y-shaped balloon comprising a proximal arm defining a proximal diameter, a first distal arm defining a first distal diameter, and a second distal arm defining a second distal diameter, where the proximal diameter and the second distal diameter are substantially equivalent and the proximal diameter is at least 1.2 times larger than the first distal diameter.

12. A balloon catheter, as in claim 11, further comprising the shaft defining an inflation lumen in fluid communication with an interior chamber of the Y-shaped balloon.
